(19) United States
(12) Patent Application Publication
(10) Pub. No.: US 2016/0331593 A1
(43) Pub. Date: Nov. 17, 2016

(54) ARTICLE TO BE APPLIED AGAINST THE SKIN, MUCOUS MEMBRANES OR A WOUND

(71) Applicants: COMMISSARIAT A L'ENERGIE ATOMIQUE ET AUX ENERGIES ALTERNATIVES, Paris (FR); URGO RECHERCHE INNOVATION ET DEVELOPPEMENT, Chenove (FR)

(72) Inventors: Frédéric REVOL-CAVALIER, Seyssins (FR); Mouna MESSAOUD, Grenoble (FR); Julien STEINBRUNN, Messigny et Vantoux (FR)

(73) Assignees: COMMISSARIAT A L'ENERGIE ATOMIQUE ET AUX ENERGIES ALTERNATIVES, Paris (FR); URGO RECHERCHE INNOVATION ET DEVELOPPEMENT, Chenove (FR)

(21) Appl. No.: 15/106,462

(22) PCT Filed: Dec. 11, 2014
(86) PCT No.: PCT/IB2014/066789

§ 371 Date: Jun. 20, 2016

(30) Foreign Application Priority Data
Dec. 19, 2013 (FR) 1363022

(51) Int. Cl. A61F 13/00

(52) U.S. Cl. CPC A61F 13/00055; A61F 13/00029; A61F 13/00042; A61F 13/00046; A61F 2013/00268; A61F 2013/00263

(57) ABSTRACT

An article to be applied against the skin, mucous membranes or a wound, in order to absorb a liquid, such as an exudate, the article including: a distal absorbent layer; one or more visual indicators that change appearance upon contact with the liquid, the visual indicator(s) being in contact with the distal absorbent layer; an interface intended to come into contact with the skin or mucous membranes; at least one proximal absorbent layer located adjacent to the interface between the interface and the distal absorbent layer; and at least one layer forming a water barrier located between the proximal absorbent layer and the distal absorbent layer and provided with at least one passage through which the liquid that has passed through the proximal absorbent layer can reach the distal absorbent layer.

The present invention relates to articles to be applied to the skin or the mucous membranes and more particularly, but not exclusively, to dressings.

There is a need to know the degree of filling of an absorbent article, especially of a dressing applied to a weakly exuding wound, in order to be able to decide whether or not to replace it, in view of using it as best as possible.

Indeed, the fact of changing a dressing too frequently may lead to a poor progression of the wound healing process, by disruption of the healing kinetics, but also to an increased risk of bacterial contamination.

However, it is often advised to change a dressing frequently, in order to avoid any prolonged contact with a medium that is too moist for the person, irrespective of the patient considered. Yet the degree of exudation of a wound depends a lot on the patient and proves difficult to quantify beforehand.

In the case where the liquid to be absorbed is the urine of a young infant and the article is a diaper, there is also an advantage in keeping the article in place until it is saturated, so as to reduce the amount of articles used and also the work required for the changing of the article by the caregiver or by the parents.

The invention aims to further improve absorbent articles to be applied to the skin or the mucous membranes in order to be able to use them under conditions that are as close as possible to optimum.

International application WO 2010/147533 describes a dressing which is not designed to indicate its degree of filling.

International application WO 2012/080865 relates to a diaper intended for an infant, which comprises a ventilation layer that makes it possible to reduce any wet contact at the interface with the skin of the infant and to distribute the liquids toward an adjacent absorbent layer; the structure comprises a distribution layer that makes it possible to homogenize the liquid over the whole of its surface. The application describes a two-dimensional printed outer pattern, optionally comprising a colored indicator, and that makes it possible to visualize the presence of secreted urine. The colored indicator does not make it possible to precisely know the degree of filling of the article by the liquid emitted, being essentially binary and making it possible to visualize whether or not urine is present.

International application WO 2013/114273 describes an absorbent article with a skin-contact face, comprising two wicking layers separated by a hydrophobic barrier film having at least one opening, an absorbent layer and an electrical means for detecting liquids comprising a network of electrodes. The structure described in that document makes it possible to visualize, by means of an electronic display, the degree of filling of a dressing for example. The hydrophobic barrier film preferably comprises a single perforation, located in the same axis as the underlying wound.

The detection device described in that document is too expensive and complex to be able to be integrated into single-use articles for mass distribution such as dressings, diapers or else intimate hygiene products.

The present invention aims to provide an absorbent article, the filling of which preferentially takes place relatively uniformly, and that has at least one means for visualizing the progression of this filling.

One subject of the invention is thus, according to one of its aspects, an article to be applied to the skin, the mucous membranes or a wound, in order to absorb therein a liquid, especially an exudate, comprising:

a distal absorbent layer,

one or more visual indicators that change appearance in the event of contact with the liquid, this or these visual indicator(s) being in contact with the distal absorbent layer,

an interface intended to contact the skin or the mucous membranes,

between the interface and the distal absorbent layer, at least one proximal absorbent layer adjacent to the interface,

between the proximal absorbent layer and the distal absorbent layer, at least one layer forming a barrier to water, provided with at least one passage enabling the liquid that has penetrated the proximal absorbent layer to reach the distal absorbent layer.

Barrier-Forming Layer

Preferably, the barrier-forming layer consists of a film of an impermeable material, especially a film of a thermoplastic material, in particular of polyurethane, for example a film such as that sold by the company PLASTO under the designation P1166.

Preferably, the layer forming a barrier to water is permeable to water vapor. Thus, the barrier-forming layer preferably has a water vapor permeability greater than or equal to 500 g/m²/24 h, measured according to the NF EN 13726-2 standard. Generally, permeability to a gas, or even to water vapor, enables a better aeration of the wound.

The aforementioned passage is preferably off-center. This makes it possible to obtain a better distribution of the liquid within the article, by forcing the liquid to be distributed within the proximal absorbent layer before passing into the distal absorbent layer. In particular, this tends to prevent the liquid absorbed from being distributed around a single axis, as in the case where there would be a central passage located opposite the source of the liquid, and this enables a finer indication of the filling level of the article by the visual indicator(s) in contact with the distal absorbent layer.

The passage is thus, preferably, unique, which makes it possible to better anticipate and control the distribution of the liquid.

Preferably, the passage(s) each have a larger transverse dimension between 100 μm and 1 μm, better still between 100 μm and 5 mm. This or these passage(s) may have a circular cross section.

The thickness of the barrier-forming layer may be between 20 μm and 100 μm.

The barrier-forming layer may be unique; as a variant, the article comprises several impermeable layers, for example laminated together.

Absorbent Layers

The distal and proximal absorbent layers each preferably have a water absorption capacity greater than or equal to 500 g/m², better still greater than or equal to 800 g/m². They may comprise or consist of any material capable of storing the liquids, such as for example the materials used in field of hygiene and dressings.

The proximal or distal absorbent layer preferably comprises a hydrophilic porous foam, preferably a polyurethane foam, like for example that sold by Laboratoires URGO under the name Cellosorb® or else that known under the name MCOF.03 and sold by the company Advanced Medical Solutions (AMS).

The proximal absorbent layer is a storage layer for the liquid absorbed.

The material of the proximal absorbent layer and its thickness are preferably chosen so that the water absorption capacity of the proximal absorbent layer is greater than the water absorption capacity of the distal absorbent layer.

The thickness of the proximal absorbent layer is for example between 1 and 5 mm.

The water absorption capacity of the proximal absorbent layer is preferably greater than that of the distal absorbent layer by a factor of 2, better still at least 5 times greater. This makes it possible to avoid creating a pumping effect and to retain most of the liquid in the proximal absorbent layer. In this way, the proximal absorbent layer is filled before the distal absorbent layer is completely filled.

The role of the distal absorbent layer is to detect the filling level of the article, not to store the majority of the liquid absorbed. In particular, it is desirable not to saturate the distal absorbent layer too quickly and not to react all the visual indicators while the proximal absorbent layer is not completely filled.

The proximal and distal absorbent layers may also comprise materials based on a superabsorbent polymer (SAP), such as for example the absorbent nonwovens incorporating SAP particles commonly used in the hygiene field, the absorbent textiles such as for example nonwovens based on viscose, rayon or cellulose, such as for example wadding, or hydrogels.

Use is preferably made of nonwovens obtained by the dry production method, known as the “airlaid” method, which contain SAP particles and in particular between 20% and 60% by weight of SAP relative to the total weight of nonwoven.

The proximal absorbent layer may thus be formed by a nonwoven superabsorbent material, for example such as that sold by the company EAM Corporation under the reference Novathin®. This gives it a high absorption capacity.

According to one variant, use is made, as SAP-based material, of a material consisting of two cellulosic webs, incorporated between which are particles of superabsorbent polymers, alone or in combination with binders.

According to another variant, use is made of a material based on SAP fibers, alone or in combination with non-absorbent fibers. Preferably, this material is in the form of a nonwoven.

Visual Indicators

Preferably, the article comprises a plurality of discrete visual indicators. This makes it possible to locate the presence of liquid at various locations of the distal absorbent layer, and thus to refine the quantification of the liquid absorbed by the article. An article is thus provided that enables a progressive visualization of its degree of filling, which gives information representative of the saturation level of the article at any moment. These indicators are distributed in the distal absorbent layer, or at the outer surface of the latter, so that they are visible when the article is applied against the skin.

Besides the advantage presented for a dressing, an article according to the invention makes it possible to define the volume of urinary secretion emitted by a young infant, owing to the visualization of the saturation level of the absorbent article. This may facilitate the analysis of the degree of dehydration of the infant.

The visual indicators are preferably positioned with a constant spacing in at least one direction, for example in the form of a grid pattern, especially with a spacing of between 5 mm and 2 cm between the centers of two indicators.

The visual indicators consist for example of pellets deposited on the distal absorbent layer, but may also be deposited by printing, by coating, or in the form of a layer having differences in concentration of a compound that produces a color in contact with water.

The visual indicators may be separated from one another by air or by a hydrophobic material that may help to keep the indicators in place, where appropriate, especially when they are pellets. Each pellet may be in contact with a porous and water-permeable confining matrix.

The visual indicators are for example colored indicators capable of changing color according to the degree of hydration of the portion of distal absorbent layer in contact therewith.

They may also be pellets of two colorless compounds, at least one of which is soluble in the liquid absorbed, which react together to form a colored product when at least one dissolves in the presence of liquid. As a variant, the compound(s) used to produce each visual indicator are initially colored, and it is the product formed after coming into contact with the liquid that is colorless or changes color.

They may also be one or more acid-base compounds having a substantially neutral pKa, the acid form of which does not have the same color as the basic form. This or these compound(s) may be coupled to a weak acid and/or a weak base that dissolves in the presence of liquid. They may also be one or more slightly oxidizing and/or reducing compounds.

The color of the visual indicators is preferably generated by absorption in the visible spectrum, so as to enable the evaluation of the degree of filling of the article without having to resort to an additional apparatus, such as an ultraviolet lamp.

The color produced by a visual indicator may correspond to various shades as a function of the amount of liquid absorbed by the article, which enables a progressive monitoring of the degree of filling of the article. For example, the color may change from white to light red, then from light red to vermilion, from vermilion to blood red, and from blood red to burgundy, as a function of the degree of water saturation. An example of a visual indicator, that takes various shades depending on the amount of liquid absorbed, is known under the reference HB Fuller Full-Care 9390.

The number of visual indicators ranges for example from 1 to 100.

The article may comprise graduations, for example printed graduations, which are associated with certain visual indicators at least and that give information on the corresponding degree of filling.

Other Layers

The interface with the skin or the mucous membranes may be defined by a contact layer, comprising for example a textile mesh. The contact layer is preferably micro-adherent. For example, it is a textile mesh coated with a gel, as described in document WO 2012/40378 or WO 2012/40377.

The expression “micro-adherent contact layer” is understood to mean that it makes it possible to be attached temporarily to the wound, this layer being able to be removed without the structure of the wound or of the perilesional skin being damaged, to the extent that said layer is repositionable and facilitates nursing care.

The article may comprise, between the contact layer and the proximal absorbent layer, at least one first distribution layer.

Indeed, it is desirable for the liquid to be as well distributed as possible, in order to be able to store more liquid in the proximal absorbent layer while avoiding a reflux of liquid toward the skin, the wound or the mucous membranes in contact with the article.

In addition, it is desirable for the liquid to be distributed uniformly in order to be able to reliably evaluate the amount of liquid absorbed, by means of the amount of liquid passing into the distal absorbent layer.

The article may also comprise, between the proximal absorbent layer and the impermeable layer, a second distribution layer. This makes it possible to further improve the distribution of the liquid in the proximal absorbent layer.

The absorption capacity of the distribution layers is relatively low with respect to that of the proximal absorbent layer, preferably between 5 and 10 times lower.

In the case where the article comprises a second distribution layer in contact with the barrier-forming layer and the proximal absorbent layer, it is desirable for the liquid not to be able to pass directly from the second distribution layer to the distal absorbent layer, but circulates from the second distribution layer to the proximal absorbent layer, then from the proximal absorbent layer to the distal absorbent layer. For this purpose, the second distribution layer may be rendered hydrophobic locally, especially by coating with an occlusive material, for example a thermoplastic material, for example PVC optionally dissolved in an appropriate solvent, such as acetonitrile, at an area located at the periphery of the passage(s). As a variant, the second distribution layer has a hole facing each of the passages. The cross section of each of these holes is preferably greater than that of the corresponding passage.

The distribution layer(s) may consist of materials that enable the spreading of the liquids by transverse or longitudinal diffusion and/or both. Such layers are for example made of hydrophilic textile or nonwoven materials based on polyester or polyolefin fibers. By way of example, mention may be made of the products sold respectively by the companies ORSA and FREUDENBERG under the names Jettex® 1205c and Evolon®.

The article may also comprise an outer protective layer, preferably that leaves the visual indicator(s) visible and is thus non-opaque, that is superimposed on the distal absorbent layer. This outer protective layer is preferably impermeable to water, and has a water vapor permeability greater than or equal to 500 g/m²/24 h. Such outer protective layers are commonly used in the production of dressings and consist for example of polyurethane films such as the films sold by the company Exopack Advanced Coating under the name INSPIRE. This protective layer is preferably translucent.

The protective layer may be joined to the rest of the article by means of an adhesive, preferably a discontinuous adhesive, so as not to affect the permeability of the layer to water vapor.

The article is preferably a dressing, especially a sterilely packaged dressing.

The invention will be able to be better understood on reading the detailed description that follows of nonlimiting exemplary embodiments thereof, and on examining the appended drawings: in which:

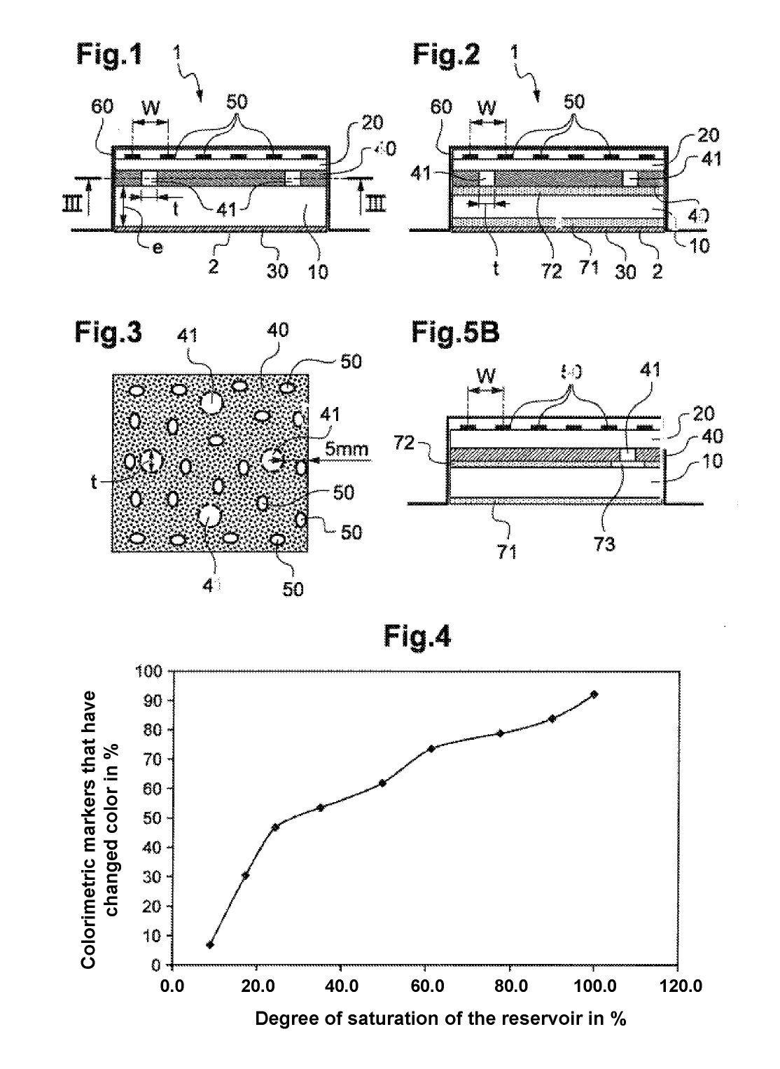

FIG. 1 is a schematic and partial cross-sectional view of an article produced in accordance with the invention,

FIG. 2 is a view analogous to FIG. 1 of an embodiment variant,

FIG. 3 is a cross-sectional view along of the article from FIG. 1,

FIG. 4 represents the change in the amount of colorimetric marker that has changed color as a function of the degree of saturation of the reservoir of an article according to the invention,

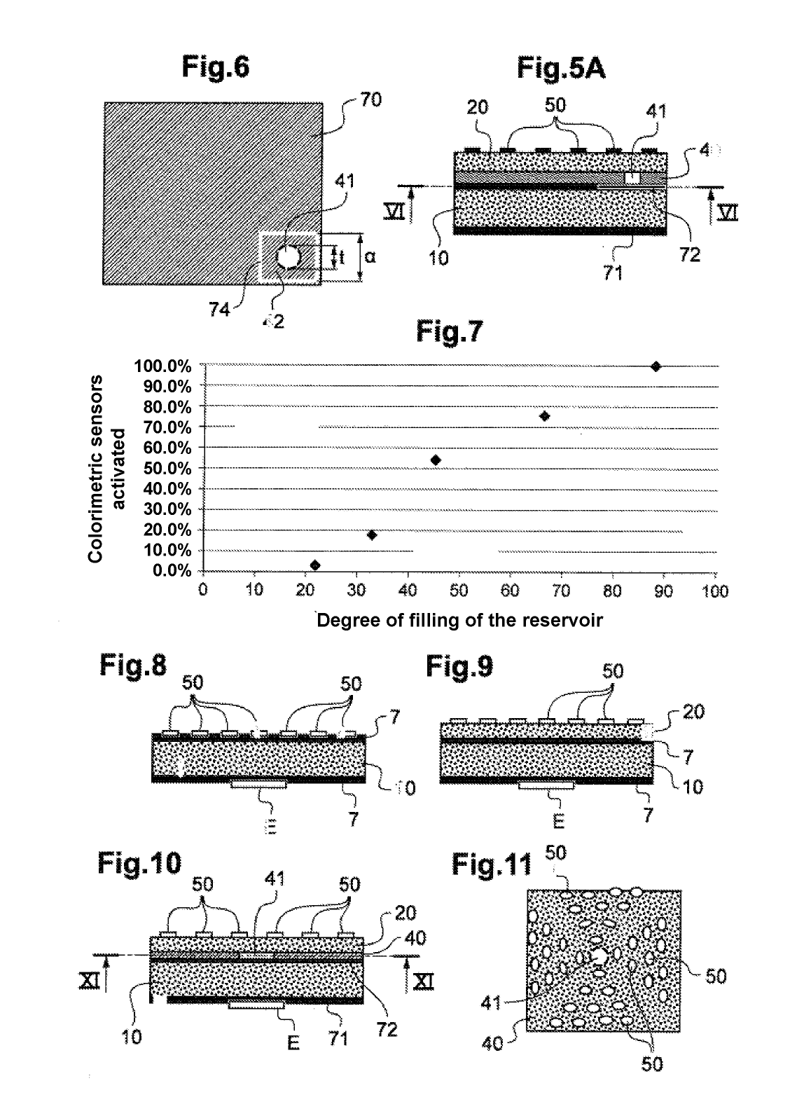

FIGS. 5A and 5B are views analogous to FIGS. 1 and 2 of other variants of articles according to the invention,

FIG. 6 is a cross-section along VI-VI of the article from FIG. 5,

FIG. 7 represents a curve analogous to the one from FIG. 4 in the case of another article according to the invention,

FIGS. 8 to 10 are views analogous to FIG. 1 of variants used to carry out comparative tests, and

FIG. 11 is a cross-section along XI-XI from FIG. 10.

In the figures, the actual proportions of the various constituent elements have not always been respected, for the sake of clarity of the drawing. Certain elements have not been represented in contact, whereas they are actually in contact. Furthermore, certain constituents, for example adhesives, were able to not be represented.

The absorbent article 1 according to the invention, represented in FIG. 1, is to be applied via a proximal face 2 to healthy or damaged skin, and/or to mucous membranes. It is preferably a dressing, but the description that follows also holds true for another article, such as a diaper or an intimate hygiene article.

The article 1 comprises one or more absorbent layers capable of accumulating the liquid received by the proximal face 2, for example the exudate from a wound.

The article 1 thus comprises, as illustrated, a first absorbent layer 10, referred to as the proximal absorbent layer or reservoir, which extends parallel to the proximal face 2, in both directions, namely the length of the article and its width when the article has an elongated shape.

The proximal absorbent layer 10 extends over a thickness e which is preferably constant and greater than or equal to 1 mm.

The contact with the skin or the mucous membranes may be defined by an interface layer 30, the proximal face 2 of which extends beneath the proximal absorbent layer 10.

The article 1 also comprises a second absorbent layer 20, referred to as the distal absorbent layer, which preferably extends over the same area as the proximal absorbent layer 10.

An intermediate barrier-forming layer 40, which is impermeable to water, extends between the proximal 10 and distal 20 absorbent layers. This intermediate layer 40 is provided with at least one passage 41 enabling the liquid contained in the proximal layer 10 to reach the distal layer 20. This passage is produced for example in the form of a hole through the material of the intermediate layer 40.

The intermediate layer 40 may comprise several passages 41, positioned for example as illustrated in FIG. 3, each at the mid-length point of a side, and closer to the side than to the center of the article.

The passage(s) 41 may all have the same largest transverse dimension t. In particular, the passage(s) 41 may have circular cross sections of diameter t between 100 μm and 5 mm.

The passages 41 may observe, in their positioning, an axial symmetry, but other arrangements are possible.

The article 1 comprises a plurality of visual indicators 50, that are visible when the article 1 is in place on the body, in order to enable the observer to evaluate the degree of filling of the article 1 and the possible need to replace it.

These indicators 50 are preferably, as illustrated, distributed discretely at the surface of the distal absorbent layer 20, for example according to a matrix arrangement as illustrated in FIG. 3.

The distribution of the indicators 50 may be carried out as a function of the positioning of the passage(s) 41 passing through the impermeable layer 40, so that the number of visual indicators 50 that have changed appearance following the presence of liquid is as faithfully as possible representative of the degree of filling of the article.

The distribution of the indicators 50 may also be carried out according to a grid pattern with a constant spacing W between the indicators in at least one direction. The spacing W is for example between 5 mm and 2 cm, especially of the order of 1 cm.

The article 1 is advantageously covered with an outer protective layer 60, that protects the distal absorbent layer 20 from the outside environment.

The protective layer 60 is for example made of a thermoplastic material, especially polyurethane, and preferably has a water vapor permeability of greater than 500 g/m²/24 h, so as not to prevent the evaporation of the liquid stored and respiration of the skin.

The protective layer 60 is joined to the rest of the article by any appropriate means, preferably by means of a discontinuous adhesive, which is not represented.

During the use of the article 1, the liquid that fills the proximal absorbent layer 10 reaches, via the passages 41, the distal absorbent layer 20. The visual indicators 50 change appearance as the liquid extends into the distal absorbent layer 20.

In order to render the distribution of the liquid uniform, the article 1 may comprise distribution layers 71 and 72 respectively located underneath and on top of the proximal absorbent layer 10, as illustrated in FIG. 2.

The role of the distribution layers 71 and 72 is not to store the liquids but to promote the spreading thereof.

The distribution layers 71 and 72 are preferably less absorbent, on the one hand, than the proximal absorbent layer 10 and, on the other hand, than the distal absorbent layer 20.

The distribution layers 71 and 72 thus have, preferably, a water absorption capacity that is less than 500 g/m².

Preferably the thickness of each of these layers 71 and 72 is between 50 and 500 μm, preferably between 250 μm and 500 μm.

Preferably again, each distribution layer 71 or 72 comprises or consists of a layer of a hydrophilic material.

Use may be made, in order to produce the distribution layers 71 and 72, of materials based on absorbent fibers of plant origin, such as viscose, cellulose or derivatives thereof. These materials may be in the form of knit fabrics, woven fabrics or nonwoven fabrics, obtained by an airlaid route or by a wetlaid route, like papers.

In order to evaluate the performance of an article 1 according to the invention, the degree of saturation of the proximal absorbent layer 10 was measured as a function of the number of indicators 50 that were activated, for an article having the structure represented in FIG. 2.

The results are reported in FIG. 4. In this example, the interface 30 consists of a nonwoven of Bérotex® type, the absorbent layers 10 and 20 consist of polyurethane foam and have respective thicknesses of 4 and 2 mm, the impermeable layer 40 consists of a polyurethane film, and the article has two distribution layers 71 and 72 in a hydrophilic nonwoven of Berkshire DR870® type. The indicators 50 used are colored indicators of HB Fuller Full-Care 9390® type. The outer protective layer 60 is a polyurethane film, optionally coated with an adhesive mass such as an acrylic adhesive, or a silicone.

The liquid supply flow rate was set at 100 μl/min onto a fritted disk having a diameter of 3 cm, so as to simulate an average-size wound. The dressing is of square shape, having an area of 81 cm². An overpressure of 15 mbar with respect to atmospheric pressure is applied to the dressing.

The proximal absorbent layer was weighed regularly so as to establish a link between the number of indicators that were activated and the saturation level of this layer.

Photographs of the indicators were taken regularly in order to evaluate the number of indicators that were activated. It is seen in FIG. 4 that the progression of the number of indicators that have changed appearance is an indicator of the degree of saturation.

The exemplary embodiments represented in FIGS. 5A, 5B and 6 are now addressed.

In order to ensure that the liquid emitted is transferred into the distal absorbent layer 20 solely through the proximal absorbent layer 10, and not through the second transfer layer 72 located on top of the proximal absorbent layer 10, the distribution layer 72 may be coated with a barrier material 42 from the second distribution layer 72 level with the passage 41, as illustrated in FIG. 5A. This is for example PVC dissolved in acetonitrile, a Sylgar 184 PDMS silicone from the company Dow Corning®, or a polymerizable resin of Hoechst AZ4620, Hoechst AZ4562, Shipley 1400-17, Shipley 1400-27 or Shipley 1400-37 type. The barrier material 42 blocks the transfer of the liquid into the distal absorbent layer 20.

This makes it possible to delay the activation of the first colored indicators and to refine the measurement of the filling for higher saturation levels of the proximal absorbent layer.

In the variant illustrated in FIG. 5B, the second distribution layer 72 comprises a hole 73 facing the passage 41 and its periphery, so as to prevent the direct propagation of the liquid from the distribution layer 72 to the distal absorbent layer 20 through the passage 41.

It is seen in FIG. 6 that the passage 41 may be unique and located in the vicinity of a corner 74 of the article, being closer to this corner than to the center of the article. FIG. 7 again plots a progression similar to FIG. 4, in the case where the passage 41 is off-center, as in FIG. 6. It is observed that for the low degrees of filling, especially less than 30%, the number of indicators that have changed appearance is smaller when the passage is off-center. The fact of moving the passage of the liquid off-center delays the activation of the first indicators.

The barrier material 42 extends for example in the direction of the width and of the length of the article over a distance d at least double the largest dimension t of the passage 41.

Comparative Tests

The configurations illustrated in FIGS. 8 to 11 represent structures that aim to demonstrate certain advantages provided by distinctive embodiment features.

The structure represented in FIG. 8 corresponds to an article not in accordance with the invention comprising only a single absorbent layer 10, positioned between and in contact with two lower 71 and upper 72 distribution layers. The lower distribution layer 71 is in contact with a liquid-emitting source E, and the upper distribution layer 72 bears a plurality of visual indicators 50. This configuration, which is characterized especially by the absence of the impermeable layer, results in too early an activation of the indicators 50.

The structure illustrated in FIG. 9 differs from that from FIG. 8 by the presence of a distal absorbent layer 20, added between the upper distribution layer 72 and the visual indicators 50. This configuration enables a better monitoring of the filling of the proximal absorbent layer 10. However, the liquid begins to be distributed between the two absorbent layers 10 and 20 as soon as the liquid reaches the upper distribution layer 72. This distribution accelerates the filling of the distal absorbent layer 20, which has the result of activating the visual indicators 50 too rapidly, in a manner not representative of the degree of filling of the proximal absorbent layer 10. This highlights the advantage of the barrier-forming layer 40 of a structure according to the invention.

In FIG. 10, which corresponds to a structure according to the invention, a barrier-forming layer 40 of a hydrophobic material having a passage 41 at its center is added to the structure from FIG. 9, between the upper distribution layer 72 and the distal absorbent layer 20. This structure makes it possible to better monitor the filling of the proximal absorbent layer 10. However, the central position of the passage 41 enables liquid to pass into the distal absorbent layer before the proximal absorbent layer is completely wetted: this configuration tends to induce an axial distribution of the liquid absorbed and an early activation of the indicators 50. Revealed in FIG. 11 is the distribution of the visual indicators 50 in this configuration. The test corresponding to FIG. 10 reveals the advantage of an off-center positioning of the passage(s) 41 when the source of fluid is central.

The invention is not limited to the examples which have been described. In particular, the various layers may comprise materials other than those mentioned, especially selected from those usually used in the production of dressings, diapers, intimate hygiene articles, and absorbent articles in general. The arrangement and organization of the various passages 41 may be adapted as a function of the geometry of the article and of the expected positioning of the source of liquid in order to optimize as best as possible its performance. Similarly, the choice of the visual indicators and their distribution may be modified without departing from the field of the invention.

The term “comprises a” is synonymous with “comprises at least one”, unless otherwise indicated.

1.-21. (canceled)

22. An article to be applied to the skin, the mucous membranes or a wound, in order to absorb therein a liquid comprising:
a distal absorbent layer,
a plurality of discrete visual indicators that change appearance in the event of contact with the liquid, this or these visual indicator(s) being in contact with the distal absorbent layer,
an interface intended to contact the skin or the mucous membranes,
between the interface and the distal absorbent layer, at least one proximal absorbent layer adjacent to the interface,
between the proximal absorbent layer and the distal absorbent layer, at least one layer forming a barrier to water provided with at least one passage enabling the liquid that has penetrated the proximal absorbent layer to reach the distal absorbent layer.

23. The article as claimed in claim 22, said at least one passage being off-center.

24. The article as claimed in claim 22, the passage being unique.

25. The article as claimed in claim 22, the passage(s) each having a larger transverse dimension (t) between 100 μm and 1 cm.

26. The article as claimed in claim 25, the visual indicators being positioned with a constant spacing in at least one direction

27. The article as claimed in claim 26, the visual indicators being positioned with a spacing (W) between 5 mm and 2 cm.

28. The article as claimed in claim 22, the visual indicator(s) consisting of pellets deposited on the distal absorbent layer.

29. The article as claimed in claim 22, the visual indicators being separated from one another by a hydrophobic material.

30. The article as claimed in claim 22, the distal absorbent layer comprising a hydrophilic porous foam.

31. The article as claimed in claim 22, the barrier-forming layer consisting of a film of an impermeable material.

32. The article as claimed in claim 22, the proximal absorbent layer comprising a foam of a hydrophilic porous material.

33. The article as claimed in claim 22, the water absorption capacity of the proximal absorbent layer being greater than that of the distal absorbent layer.

34. The article as claimed in claim 22, the interface being defined by a contact layer.

35. The article as claimed in claim 34, comprising, between the contact layer and the proximal absorbent layer, at least one first distribution layer.

36. The article as claimed in claim 35, comprising, between the proximal absorbent layer and the barrier-forming layer, a second distribution layer.

37. The article as claimed in claim 36, the second distribution layer being rendered hydrophobic locally over an area located facing the periphery of the passage(s).

38. The article as claimed in claim 36, the second distribution layer having a hole facing the passage(s).

39. The article as claimed in claim 35, the distribution layer(s) comprising a hydrophilic textile or nonwoven.

40. The article as claimed in claim 22, the barrier-forming layer having a water vapor permeability greater than or equal to 500 g/m²/24 h.

41. The article as claimed in claim 22, comprising a protective layer, that is superimposed on the distal absorbent layer, is impermeable to water.

42. The article as claimed in claim 41, the protective layer having a water vapor permeability greater than 500 g/m²/24 h.

43. The article as claimed in claim 41, the protective layer being translucent.

44. The article as claimed in claim 41, the protective layer being joined to the article by means of an adhesive.
